(19) United States
(12) Patent Application Publication
(10) Pub. No.: US 2024/0043376 A1
(43) Pub. Date: Feb. 8, 2024

(54) COMPOSITION

(71) Applicant: Innospec Active Chemicals LLC, High Point, NC (US)

(72) Inventor: Phillip Loraine Cotrell, Salisbury, NC (US)

(21) Appl. No.: 18/223,664

(22) Filed: Jul. 19, 2023

Related U.S. Application Data
(60) Provisional application No. 63/392,591, filed on Jul. 27, 2022.

(30) Foreign Application Priority Data
Sep. 6, 2022 (GB) 2212972.0

(51) Int. Cl. C07C 309/21; C07C 303/22

(52) U.S. Cl. CPC C07C 309/21; C07C 303/22

(57) ABSTRACT

A N-acylated taurine composition comprising:
(a) a first N-acylated taurine compound of the formula (IA) or a salt thereof:

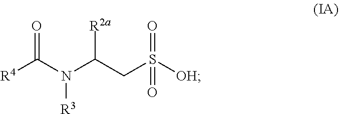

and (b) a second N-acylated taurine compound of the formula (IB) or a salt thereof:

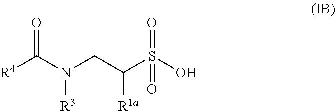

wherein
R^{1a} and R^{2a} are C₁₋₄ alkyl, wherein R^{1a} and R^{2a} are the same;
R³ is C₁₋₆ alkyl, C₂₋₆ alkenyl or C₁₋₆ alkyl substituted with an aryl group; and
R⁴ is C₄₋₂₅ alkyl or C₄₋₂₅ alkenyl, wherein the C₄₋₂₅ alkyl or C₄₋₂₅ alkenyl is optionally substituted by hydroxy.

### CROSS-REFERENCE TO RELATED APPLICATIONS

This non-provisional patent application claims priority to U.S. provisional patent application No. 63/392,591, filed Jul. 27, 2022 and to British patent application GB 2212972.0, filed Sep. 6, 2022; each of which is individually incorporated herein by reference in its entirety.

### FIELD OF THE INVENTION

The present invention relates to a N-acylated taurine composition and a method of preparing the N-acylated taurine composition. The N-acylated taurine composition comprises an isomeric mixture of N-acylated taurine compounds. The present invention also provides an N-acylated taurine compound and a taurine composition. The N-acylated taurine composition and compound may be useful in a broad range of applications.

### BACKGROUND OF THE INVENTION

Taurate compounds (such as N-methyl taurate compounds) are known as anionic surfactants and are widely used as foaming and cleansing agents in a wide variety of applications. Such applications include personal care, home care, industrial and agricultural applications. The taurate compounds are also hydrolytically stable over a wide pH range, such as a pH range of 2 to 13. This hydrolytic stability makes the taurate compounds desirable for use in applications that require stability over a broad range of pH values.

The taurate compounds are typically provided as soft or firm pastes or as viscous liquids, such that they require storing in hot rooms and/or heating before use. This makes the compounds difficult and expensive to store and/or to handle, especially on large scales.

There is therefore a need for alternative taurate type compounds and compositions that provide the advantageous properties of the known taurate compounds but which are easier to store and/or handle at ambient temperatures.

### BRIEF DESCRIPTION OF THE INVENTION

According to a first aspect of the present invention there is provided a N-acylated taurine composition comprising:
(a) a first N-acylated taurine compound of the formula (IA) or a salt thereof:

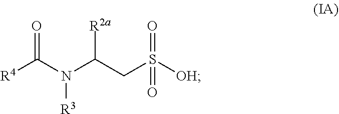

and (b) a second N-acylated taurine compound of the formula (IB) or a salt thereof

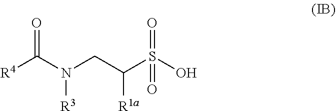

wherein
R^{1a} and R^{2a} are C₁₋₄ alkyl, wherein R^{1a} and R^{2a} are the same;
R³ is C₁₋₆ alkyl, C₂₋₆ alkenyl or C₁₋₆ alkyl substituted with an aryl group; and
R⁴ is C₄₋₂₅ alkyl or C₄₋₂₅ alkenyl, wherein the C₄₋₂₅ alkyl or C₄₋₂₅ alkenyl is optionally substituted by hydroxy.

According to a second aspect of the present invention, there is provided a method of preparing the N-acylated taurine composition according to the first aspect, the method comprising reacting a taurine composition comprising a first taurine compound of the formula (IIA) or a salt thereof and a second taurine compound of the formula (IIB) or a salt thereof:

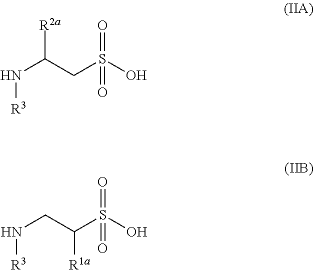

with a fatty acid of the formula R⁴—C(O)OH or a reactive derivative thereof, wherein R^{1a}, R^{2a}, R³ and R⁴ are each as defined in the first aspect of the present invention, to provide the N-acylated taurine composition.

According to a third aspect of the present invention there is provided a N-acylated taurine compound of the formula (IC) or a salt thereof:

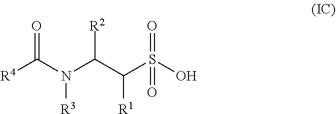

wherein:
R¹ and R² are each independently selected from H or C₁₋₄ alkyl, provided that one of R¹ and R² is H and the other of R¹ and R² is C₁₋₄ alkyl;
R³ is C₁₋₆ alkyl, C₂₋₆ alkenyl, or C₁₋₆ alkyl substituted with an aryl group; and
R⁴ is C₄₋₂₅ alkyl or C₄₋₂₅ alkenyl wherein the C₄₋₂₅ alkyl or C₄₋₂₅ alkenyl is optionally substituted by hydroxy.

According to a fourth aspect of the present invention there is provided a taurine composition comprising a first taurine compound of the formula (IIA) or a salt thereof and a second taurine compound of the formula (IIB) or a salt thereof:

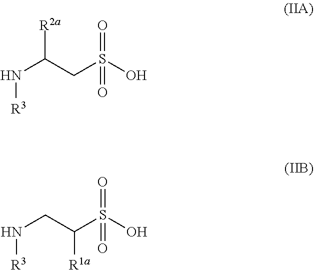

wherein
R^{1a} and R^{2a} are C₁₋₄ alkyl, wherein R^{1a} and R^{2a} are the same; and
R³ is C₁₋₆ alkyl, C₂₋₆ alkenyl or C₁₋₆ alkyl substituted with an aryl group.

### DETAILED DESCRIPTION

Unless otherwise stated, the following terms used in the specification and claims have the meanings set out below.

The terms “alkyl” and “alkenyl” include both straight and branched chain alkyl and alkenyl groups respectively.

The term “aryl” as used herein relates to an organic radical derived from an aromatic hydrocarbon by removal of one hydrogen, and includes any monocyclic, bicyclic or polycyclic carbon ring of up to 7 members in each ring, wherein at least one ring is aromatic.

As used in the specification and the appended claims, the singular forms “a”, “an,” and “the” include both singular and plural referents unless the context clearly dictates otherwise.

Throughout this specification, the term “comprising” or “comprises” means including the component(s) specified but not to the exclusion of the presence of other components. The term “consisting essentially of” or “consists essentially of” means including the components specified but excluding other components except for components added for a purpose other than achieving the technical effect of the invention. The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

As used herein, unless otherwise expressly specified, all numbers such as those expressing values, ranges, amounts of percentages may be read as if prefaced by the word “about”, even if the term does not expressly appear.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

The optional features set out herein may be used either individually or in combination with each other where appropriate and particularly in the combinations as set out in the accompanying claims. The optional features for each exemplary aspect of the invention, as set out herein are also applicable to any other aspects or exemplary aspects of the invention, where appropriate. In other words, the skilled person reading this specification should consider the optional features for each aspect or embodiment of the invention as interchangeable and combinable between different aspects of the invention.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

According to a first aspect of the present invention there is provided a N-acylated taurine composition comprising (a) a first N-acylated taurine compound of the formula (IA) or a salt thereof:

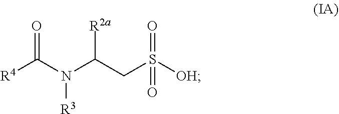

and (b) a second N-acylated taurine compound of the formula (IB) or a salt thereof:

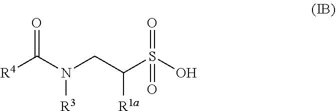

wherein
R^{1a} and R^{2a} are C₁₋₄ alkyl, wherein R^{1a} and R^{2a} are the same;
R³ is C₁₋₆ alkyl, C₂₋₆ alkenyl or C₁₋₆ alkyl substituted with an aryl group; and
R⁴ is C₄₋₂₅ alkyl or C₄₋₂₅ alkenyl, wherein the C₄₋₂₅ alkyl or C₄₋₂₅ alkenyl is optionally substituted by hydroxy.

The N-acylated taurine composition of the first aspect of the invention is advantageously a mobile, pumpable and/or pourable liquid at ambient temperature and typically at temperatures below ambient temperature, such as below 10° C., for example below 5° C. or below 0° C. The N-acylated taurine composition of the first aspect of the invention is advantageously a mobile, pumpable and/or pourable liquid at temperatures above −10° C. and below ambient temperature. Thus, the N-acylated taurine composition of the first aspect of the invention is easy to handle, store and formulate.

The N-acylated taurine composition of the first aspect of the invention is typically advantageously a substantially clear or clear liquid at ambient temperature and typically at temperatures below ambient temperature, such as below 10° C., for example below 5° C. or below 0° C. The N-acylated taurine composition of the first aspect of the invention is typically advantageously a substantially clear or clear liquid at temperatures above −10° C. and below ambient temperature. Thus, the N-acylated taurine composition of the first aspect of the invention may not discolour or impart haze to other compositions when used in combination.

The first and second N-acylated taurine compounds (or salts thereof) in the N-acylated taurine composition of the first aspect of the invention are structural isomers, such that the N-acylated taurine composition of the first aspect of the invention is an isomeric composition.

R^{1a} and R^{2a} in the compounds of the formula (IA) and (IB) or salts thereof are C₁₋₄ alkyl, wherein R^{1a} and R^{2a} are the same. Suitably, R^{1a} and R^{2a} are C₁₋₂ alkyl, wherein R^{1a} and R^{2a} are the same. Preferably, R^{1a} and R^{2a} are both methyl.

R³ in the compounds of the formula (IA) and (IB) or salts thereof is C₁₋₆ alkyl, C₂₋₆ alkenyl or C₁₋₆ alkyl substituted with an aryl group. Suitably, R³ is C₁₋₆ alkyl or C₂₋₆ alkenyl. More suitably R³ is C₁₋₆ alkyl, such as C₁₋₂ alkyl. Preferably, R³ is methyl.

R⁴ in the compounds of the formula (IA) and (IB) or salts thereof is C₄₋₂₅ alkyl or C₄₋₂₅ alkenyl, wherein the C₄₋₂₅ alkyl or C₄₋₂₅ alkenyl is optionally substituted by hydroxy. Suitably, R⁴ is C₄₋₂₅ alkyl, such as C₈₋₁₈ alkyl, wherein the C₄₋₂₅ alkyl, such as C₈₋₁₈ alkyl, is optionally substituted by hydroxy. Suitably, R⁴ is an unsubstituted C₄₋₂₅ alkyl, such as an unsubstitued C₈₋₁₈ alkyl.

R⁴ as defined herein may comprise a mixture of C₄₋₂₅ alkyl or C₄₋₂₅ alkenyl groups, for example when these groups are derived from a natural source. Examples of suitable natural sources include fatty acid mixtures obtained directly from coconut oil or palm kernel oil, or obtained after a processing step such as hydrogenation (for example to reduce amounts of unsaturated C₁₈ compounds) or “topping” (i.e. distillation of the bulk fatty acid mixture to reduce the levels of C₈ and/or C₁₀ fatty acids).

References to salts of the compounds of the formula (IA) and (IB) include any suitable salt. For example, salts of the compounds of the formula (IA) and (IB) may be in the form of a salt (IA′) and (IB′) as follows:

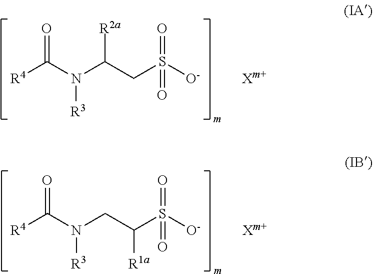

wherein R^{1a}, R^{2a}, R³ and R⁴ are as defined herein, m is 1 or 2 and X^{m +} is any suitable cation that provides charge neutrality, such as an alkali metal or alkaline earth metal cation (for example a sodium, potassium, lithium, calcium or magnesium cation) or an ammonium or substituted ammonium cation (for example a dimethyl ammonium or tetra-n-butylammonium cation). Preferably, X⁺ is a sodium cation.

Salts of N-acylated taurine compounds are otherwise known as taurates.

The first aspect of the invention may provide a N-acylated taurine composition wherein:
R^{1a} and R^{2a} are C₁₋₂ alkyl (for example methyl), wherein R^{1a} and R^{2a} are the same;
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The first aspect of the invention may provide a N-acylated taurine composition wherein:
R^{1a} and R^{2a} are both methyl;
R³ is C₁₋₂ alkyl (for example methyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The first aspect of the invention may provide a N-acylated taurine composition wherein:
R^{1a} and R^{2a} are C₁₋₂ alkyl (for example methyl), wherein R^{1a} and R^{2a} are the same;
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is unsubstituted.

The first aspect of the invention may provide a N-acylated taurine composition wherein:
R^{1a} and R^{2a} are both methyl;
R³ is C₁₋₂ alkyl (for example methyl); and
R⁴ is C₈₋₁₈ alkyl or C₈₋₁₈ alkenyl, wherein the C₈₋₁₈ alkyl or C₈₋₁₈ alkenyl is unsubstituted.

The N-acylated taurine composition of the first aspect of the invention may comprise the N-acylated taurine compounds of the formulae (IA) and (IB) (or salts thereof) in any suitable ratio. For example, the N-acylated taurine composition may comprise the first N-acylated taurine compound of the formula (IA) or a salt thereof and the second N-acylated taurine compound of the formula (IB) or a salt thereof in a molar ratio of 99:1 to 1:99, for example 99:1 to 1:1. A preferred ratio may be, for example, 99:1 to 4:1 or 9:1 to 4:1. Other preferred ratios may be 85:15 or 97:3.

According to a second aspect of the present invention there is provided a method of preparing the N-acylated taurine composition according to the first aspect of the invention, the method comprising reacting a taurine composition comprising a first taurine compound of the formula (IIA) or a salt thereof and a second taurine compound of the formula (IIB) or a salt thereof:

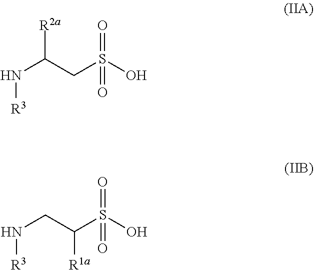

with a fatty acid of the formula R⁴—C(O)OH or a reactive derivative thereof, wherein R^{1a}, R^{2a}, R³ and R⁴ are each as defined herein, to provide the N-acylated taurine composition. Any suitable reactive derivative of R⁴—C(O)OH may be used, such as an acyl chloride R⁴—C(O)Cl.

The first taurine compound of the formula (IIA) and the second taurine compound of the formula (IIB) (or salts thereof) in the taurine composition are structural isomers, such that this represents an isomeric (taurine) mixture. For example, the taurine composition may comprise the first taurine compound of the formula (IIA) and the second taurine compound of the formula (IIB) (or salts thereof) in a molar ratio of 99:1 to 1:99, for example 99:1 to 1:1. A preferred ratio may be, for example, 99:1 to 4:1 or 9:1 to 4:1. Other preferred ratios may be 85:15 or 97:3.

The taurine composition is reacted with a fatty acid of the formula R⁴—C(O)OH or a reactive derivative thereof, wherein R^{1a}, R^{2a}, R³ and R⁴ are each as defined herein, under any suitable reaction conditions. This reaction may be conducted under any suitable reaction conditions, which depend on the particular reagents used.

For example, when the taurine composition is reacted with a fatty acid of the formula R⁴—C(O)OH, suitable reaction temperatures are from 160 to 230° C. In this case, the reaction may be conducted in the presence of a suitable catalyst. Suitable catalysts would be well known to those skilled in the art.

When the taurine composition is reacted with a reactive derivative of a fatty acid of the formula R⁴—C(O)OH, such as a fatty acid chloride R⁴—C(O)Cl, suitable reaction temperatures are from 45 to 75° C., such as from 50 to 65° C.

Any suitable reaction solvent may be used such as water or an alcohol, preferably water. The reaction is typically conducted at a pH of 9.5 to 10.5, which pH may be maintained by addition of a suitable base such as sodium hydroxide, or by addition of a suitable buffer.

The method of the second aspect of the invention may further comprise preparing the taurine composition by reacting an isethionic acid composition comprising a first isethionic acid compound of the formula (IIIA) or a salt thereof and a second isethionic acid compound of the formula (IIIB) or a salt thereof:

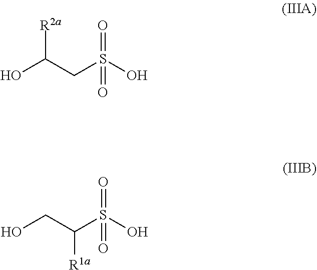

with a primary amine compound of the formula H₂NR³ to provide the taurine composition.

References to salts of the compounds of the formula (IIIA) and (IIIB) include any suitable salt. For example, salts of the compounds of the formula (IIIA) and (IIIB) may be in the form of a salt (IIIA′) and (IIIB′) as follows:

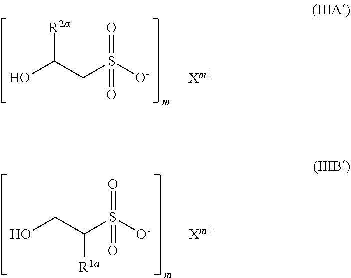

wherein R^{1a} and R^{2a} are each as defined herein, m is 1 or 2 and X^{m +} is any suitable cation that provides charge neutrality, such as an alkali metal or alkaline earth metal cation (for example a sodium, potassium, lithium, calcium or magnesium cation) or an ammonium or substituted ammonium cation (for example a dimethyl ammonium or tetra-n-butylammonium cation). Preferably, X⁺ is a sodium cation.

Thus, the method of preparing the N-acylated taurine composition according to the first aspect of the invention, may comprise:
(1) reacting an isethionic acid composition comprising a first isethionic acid compound (IIIA) or a salt thereof and a second isethionic acid compound (IIIB) or a salt thereof:

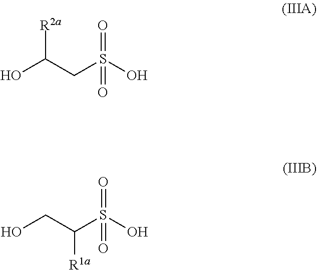

with a primary amine compound of the formula H₂NR³ to provide a taurine composition comprising a first taurine compound of the formula (IIA) or a salt thereof and a second taurine compound of the formula (IIB) or a salt thereof:

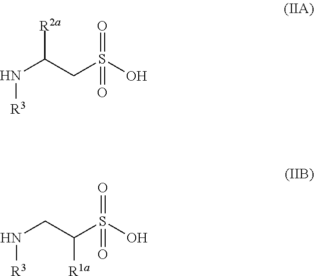

and (2) reacting the taurine composition obtained in step (1) with a fatty acid of the formula R⁴—C(O)OH or a reactive derivative thereof, wherein R^{1a}, R^{2a}, R³ and R⁴ are each as defined herein, to provide the N-acylated taurine composition.

The first isethionic acid compound (IIIA) and second isethionic acid compound (IIIB) (or salts thereof) in the isethionic acid composition are structural isomers, such that this represents an isomeric (isethionic acid) mixture. Typical molar ratios of the first isethionic acid compound (IIIA) and second isethionic acid compound (IIIB) (or salts thereof) in the isethionic acid composition may be in the range of 99:1 to 1:1, for example 99:1 to 4:1.

The isethionic acid composition comprising a first isethionic acid compound (IIIA) or a salt thereof and a second isethionic acid compound (IIIB) or a salt thereof is well known and may be obtained by any suitable means, for example as described in U.S. Pat. No. 8,105,993. Typically, the isethionic acid composition is prepared and/or supplied as a mixture of isomers and reacted without separation of the isomers.

For example, the isethionic acid composition comprising a first isethionic acid compound (IIIA) or a salt thereof and a second isethionic acid compound (IIIB) or a salt thereof may be prepared by reacting a source of bisulfite anions, such as of the formula HO—S(O)—O⁻X⁺, with an alkylene oxide of the formula (IV):

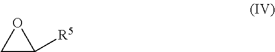

wherein R⁵ is the same as R^{1a} and R^{2a} and X⁺ is a cation as defined herein (for example wherein m is 1). This reaction may be conducted under any suitable reaction conditions as would be known to persons skilled in the art. For example, the reaction may be conducted at a pH of 4 to 10, such as 5 to 10, for example about 7. The reaction may be conducted at a temperature of 20 to 200° C., such as 30 to 95° C., for example 50 to 80° C. The reaction may be conducted at a pressure of 0 to 0.7 mPa, such as of 0.07 to 0.3 mPa.

The isethionic acid composition may be reacted with the primary amine under any suitable reaction conditions. Suitable reaction temperatures are from 200 to 280° C., such as from 220 to 260° C. Any suitable reaction solvent may be used such as water and/or an aliphatic alcohol, preferably water.

The method of the second aspect of the invention may include the step of removing excess primary amine compound of the formula H₂NR³ from the reaction product, such as after the reaction of the isethionic acid composition with the primary amine is complete.

According to a third aspect of the present invention there is provided a N-acylated taurine compound of the formula (IC) or a salt thereof:

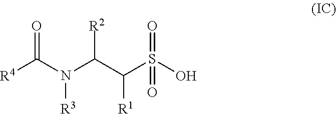

wherein:
R¹ and R² are each independently selected from H or C₁₋₄ alkyl, provided that one of R¹ and R² is H and the other of R¹ and R² is C₁₋₄ alkyl;
R³ is C₁₋₆ alkyl, C₂₋₆ alkenyl, or C₁₋₆ alkyl substituted with an aryl group; and
R⁴ is C₄₋₂₅ alkyl or C₄₋₂₅ alkenyl wherein the C₄₋₂₅ alkyl or C₄₋₂₅ alkenyl is optionally substituted by hydroxy.

R¹ and R² in the compound of the formula (IC) or a salt thereof are selected from H and C₁₋₄ alkyl, provided that one of R¹ and R² is H and the other of R¹ and R² is C₁₋₄ alkyl. Suitably, one of R¹ and R² is C₁₋₄ alkyl and the other is H. Suitably, one of R¹ and R² is C₁₋₂ alkyl and the other is H. Suitably, one of R¹ and R² is methyl and the other is H.

In one embodiment in the compound of the formula (IC) or a salt thereof, R¹ is C₁₋₄ alkyl and R² is H. Suitably, R¹ is C₁₋₂ alkyl and R² is H. Suitably, R¹ is methyl and R² is H.

In one embodiment in the compound of the formula (IC) or a salt thereof, R² is C₁₋₄ alkyl and R¹ is H. Suitably, R² is C₁₋₂ alkyl and R¹ is H. Suitably, R² is methyl and R¹ is H.

R³ in the compound of the formula (IC) or a salt thereof is C₁₋₆ alkyl, C₂₋₆ alkenyl or C₁₋₆ alkyl substituted with an aryl group. Suitably, R³ is C₁₋₆ alkyl or C₂₋₆ alkenyl. More suitably, R³ is C₁₋₆ alkyl, such as C₁₋₂ alkyl. Preferably, R³ is methyl.

R⁴ in the compound of the formula (IC) or a salt thereof is C₄₋₂₅ alkyl or C₄₋₂₅ alkenyl, wherein the C₄₋₂₅ alkyl or C₄₋₂₅ alkenyl is optionally substituted by hydroxy. Suitably, R⁴ is C₈₋₁₈ alkyl or C₈₋₁₈ alkenyl, wherein the C₈₋₁₈ alkyl or C₈₋₁₈ alkenyl, is optionally substituted by hydroxy. Suitably, R⁴ in the compound of the formula (IC) is an unsubstituted C₈₋₁₈ alkyl or an unsubstituted C₈₋₁₈ alkenyl.

R⁴ in the compound of the formula (IC) or a salt thereof may be C₄₋₂₅ alkyl, such as C₈₋₁₈ alkyl, wherein the C₄₋₂₅ alkyl, or C₈₋₁₈ alkyl, is optionally substituted by hydroxy. Suitably, R⁴ in the compound of the formula (IC) may be an unsubstituted C₄₋₂₅ alkyl, such as an unsubstituted C₈₋₁₈ alkyl

References to salts of the compound of the formula (IC) include any suitable salt. For example, salts of the compound of the formula (IC) may be in the form of a salt (IC') as follows:

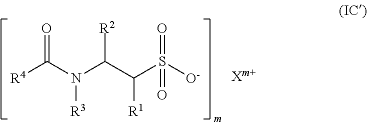

wherein R¹, R², R³ and R⁴ are each as defined herein, m is 1 or 2 and X^{m +} is any suitable cation that provides charge neutrality, such as an alkali metal or alkaline earth metal cation (for example a sodium, potassium, lithium, calcium or magnesium cation) or an ammonium or substituted ammonium cation (for example a dimethyl ammonium or tetra-n-butylammonium cation). Preferably, X⁺ is a sodium cation.

The third aspect of the invention may provide a N-acylated taurine compound of the formula (IC) or a salt thereof wherein:
R¹ and R² are each independently selected from H or C₁₋₂alkyl (for example methyl), provided that one of R¹ and R² is H the other of R¹ and R² is C₁₋₂ alkyl;
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The third aspect of the invention may provide a N-acylated taurine compound of the formula (IC) or a salt thereof wherein:
R¹ is C₁₋₄ alkyl and R² is H.
R³ is C₁₋₆ alkyl (for example C₁₋₂ alkyl);
R⁴ is C₈₋₁₈ alkyl or C₈₋₁₈ alkenyl, wherein the C₈₋₁₈ alkyl or C₈₋₁₈ alkenyl is optionally substituted by hydroxy.

The third aspect of the invention may provide a N-acylated taurine compound of the formula (IC) or a salt thereof wherein:
R¹ is C₁₋₂ alkyl and R² is H
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The third aspect of the invention may provide a N-acylated taurine compound of the formula (IC) or a salt thereof wherein:
R¹ is methyl and R² is H
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The third aspect of the invention may provide a N-acylated taurine compound of the formula (IC) or a salt thereof wherein:
R² is C₁₋₄ alkyl and R¹ is H;
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The third aspect of the invention may provide a N-acylated taurine compound of the formula (IC) or a salt thereof wherein:
R² is C₁₋₂ alkyl and R¹ is H;
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The third aspect of the invention may provide a N-acylated taurine compound of the formula (IC) or a salt thereof wherein:
R² is methyl and R¹ is H;
R³ is C₁₋₆ alkyl (for example C₁₋₂ alkyl); and
R⁴ is C₈₋₁₈ alkyl or C₈₋₁₈ alkenyl, wherein the C₈₋₁₈ alkyl or C₈₋₁₈ alkenyl is optionally substituted by hydroxy.

The N-acylated taurine compound of the third aspect of the invention may be prepared by any suitable method. For example, the N-acylated taurine compound may be isolated from a N-acylated taurine composition according to the first aspect of the invention, i.e. by isolating the compound from the composition. The N-acylated taurine compound may be prepared from an appropriate taurine compound (for example isolated from the taurine composition as defined herein) by reaction with a fatty acid or reactive derivative thereof as defined herein.

According to a fourth aspect of the present invention there is provided a taurine composition comprising a first taurine compound of the formula (IIA) or a salt thereof and a second taurine compound of the formula (IIB) or a salt thereof:

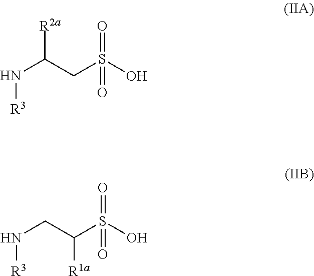

wherein
R^{1a} and R^{2a} are C₁₋₄ alkyl, wherein R^{1a} and R^{2a} are the same; and
R³ is C₁₋₆ alkyl, C₂₋₆ alkenyl or C₁₋₆ alkyl substituted with an aryl group.

The first and second taurine compounds in the taurine composition of the fourth aspect of the invention are structural isomers, such that the taurine composition of the fourth aspect of the invention is an isomeric (taurine) composition.

R^{1a} and R^{2a} in the compounds of the formula (IIA) and (IIB) (or salts thereof) are C₁₋₄ alkyl, wherein R^{1a} and R^{2a} are the same. Suitably, R^{1a} and R^{2a} are C₁₋₂ alkyl, wherein R^{1a} and R^{2a} are the same. Preferably, R^{1a} and R^{2a} are both methyl.

R³ in the compounds of the formula (IIA) and (IIB) (or salts thereof) is C₁₋₆ alkyl, C₂₆ alkenyl or C₁₋₆ alkyl substituted with an aryl group. Suitably, R³ is C₁₋₆ alkyl or C₂₋₆ alkenyl. More suitably R³ is C₁₋₆ alkyl, such as C₁₋₂ alkyl. Preferably, R³ is methyl.

References to salts of the compounds of the formula (IIA) and (IIB) include any suitable salt.

For example, under basic conditions the salts of the compounds of the formula (IIA) and (IIB) may be in the form of a sulfonate salt (IIA′) and (JIB′) (with the amine group in its free base form) as follows:

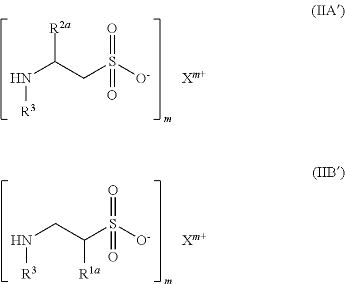

wherein R^{1a}, R^{2a} and R³ are each as defined herein, m is 1 or 2 and X^{m +} is any suitable cation that provides charge neutrality, such as an alkali metal or alkaline earth metal cation (for example a sodium, potassium, lithium, calcium or magnesium cation) or an ammonium or substituted ammonium cation (for example a dimethyl ammonium or tetra-n-butylammonium cation). Preferably, X⁺ is a sodium cation.

For example, under acidic conditions the salts of the compounds of the formula (IIA) and (IIB) may be in the form of an ammonium salt (IIA″) and (IIB″) (with a sulfonic acid group in its free acid form) as follows:

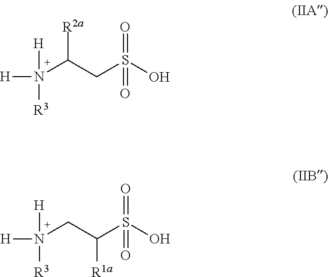

wherein R^{1a}, R^{2a} and R³ are each as defined herein.

For example, at the isoelectric point the salts of the compounds of the formula (IIA) and (IIB) may be in the form of a zwitterion (also referred to as an inner or internal salt). For example, salts of the compounds of the formula (IIA) and (IIB) may be in the form of a zwitterion (IIA′″) and (IIIB′″)as follows:

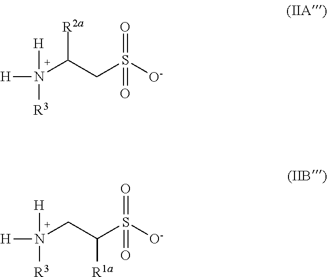

wherein R^{1a}, R^{2a} and R³ are each as defined herein.

The fourth aspect of the invention may provide a taurine composition wherein:
R^{1a} and R^{2a} are C₁₋₂ alkyl (for example methyl), wherein R^{1a} and R^{2a} are the same; and
R³ is C₁₋₆ alkyl (for example C₁₋₂ alkyl).

The fourth aspect of the invention may provide a taurine composition wherein:
R^{1a} and R^{2a} are both methyl; and
R³ is C₁₋₂ alkyl (for example methyl).

The taurine composition of the fourth aspect of the invention may comprise the taurine compounds of the formulae (IIA) and (IIB) (or salts thereof) in any suitable ratio. For example, the taurine composition may comprise the first taurine compound of the formula (IIA) or a salt thereof and the second taurine compound of the formula (IIB) or a salt thereof in a molar ratio of 99:1 to 1:99, for example 99:1 to 1:1. A preferred ratio may be, for example, 99:1 to 4:1 or 9:1 to 4:1. Other preferred ratios may be 85:15 or 97:3.

The taurine composition of the fourth aspect of the invention may be prepared by any suitable method, such as by reaction of an isethionate composition comprising a first isethionic acid compound of the formula (IIIA) and a second isethionic acid compound of the formula (IIIB) (or salts thereof) with a primary amine compound of the formula H₂NR³ as described herein in relation to the second aspect of the invention.

The compositions of the first aspect of the invention and the compound of the third aspect of the invention may be useful in a variety of applications. The present invention further provides the use of a composition according to the first aspect of the invention or a compound according to the third aspect of the invention in the formulation of a personal care, home care, industrial or agricultural product.

### BRIEF DESCRIPTION OF THE FIGURES

For a better understanding of the invention, and to show how exemplary embodiments of the same may be carried into effect reference will be made, by way of example only, to the accompanying Figures, in which:

FIG. 1 shows the physical form of a sodium cocoyl N-methyl methyl taurate composition of Example 2 at ambient temperature and a concentration of 30.4 wt % active in water; and

FIG. 2 shows the physical form of sodium cocoyl N-methyl taurate of Example 3 at ambient temperature and a concentration of 30 to 31.5 wt % active in water.

The invention will now be further described with reference to the following non-limiting examples.

### EXAMPLES

### Example 1 (Inventive)—Synthesis of Isomeric N-methyl Methyl Taurine Mixture

A 1 L stainless steel autoclave was fitted with overhead stirrer, thermocouple, pressure gauge and bursting disc. Sodium methyl isethionate (50 wt % solution in water, 156.3 g, 0.47 mol) and methylamine (40 wt % solution in water, 449.8 g, 5.79 mol) were charged. The autoclave was sealed and heated to 250° C. and held at this temperature for 5.5 hours. A pressure build-up of ˜90 bar was observed. After cooling to room temperature, the autoclave was vented. The reaction mass was discharged and concentrated on a rotary evaporator. After removal of excess methylamine, the vacuum was gradually increased to 125 mbar at a bath temperature of 70° C. to partially remove water. The partially concentrated product had an active material content of 60 wt %.

A sample of the reaction product was concentrated to dryness. ¹H and ¹³C NMR analysis (D₂O) of the dried product showed the sodium N-methyl methyl taurine product (isomeric mixture) as the major component. Based on NMR integration, the molar ratio of isomers was calculated as 97:3 (sodium 2-(methylammonium)propane-1-sulfonate to sodium 1-(methylammonium)propane-2-sulfonate).

### Example 2 (Inventive)—Synthesis of Isomeric Sodium Cocoyl N-methyl Methyl Taurate Mixture

A 1 L jacketed reactor was fitted with overhead stirrer, thermometer, pH probe and dropping funnel. N-methyl methyl taurine (60 wt % solution, 121.6 g, 0.416 mol) and water (238 g) were charged. Stirring was started and the reaction mass heated to 55° C. Cocoyl chloride (94.75 g, 0.43 mol) was charged over 3 hours via the dropping funnel; simultaneously the reaction pH was maintained in the range 9.5-10.5 by manual addition of 50 wt % aqueous NaOH solution (˜37 g overall). After completion of cocoyl chloride addition, the reaction temperature was increased to and held for 1 hour. The reaction mass was cooled to 50° C. and the pH adjusted to 7.8. Water was added to provide a final sodium cocoyl N-methyl methyl taurate content (mixture of isomers) of 30.4 wt %.

### Example 3 (Comparative)—Sodium Cocoyl N-methyl Taurate

A sample of sodium cocoyl N-methyl taurate (commercial name: Pureact® WS Conc) was obtained. This material was prepared from N-methyl taurine in analogous manner to Example 2, and had an active content of 30-31.5 wt %.

### Example 4—Physical Form Comparison of Isomeric Sodium Cocoyl N-methyl methyl Taurate Mixture and Sodium Cocoyl N-methyl Taurate

The physical form of isomeric sodium cocoyl N-methyl methyl taurate mixture (Example 2) and sodium cocoyl N-methyl taurate (Example 3) were compared at ambient temperature, for the same active concentration.

The results are shown in Table 1.

TABLE 1
Physical form comparison of isomeric sodium cocoyl N-methyl
methyl taurate mixture and sodium cocoyl N-methyl taurate

| Example | 2 (inventive) | 3 (comparative) |
|---|---|---|
| Active content (wt %) | 30.4 | 30-31.5 * |
| Non volatiles (wt %) | 40.9 | 38-43.0 * |
| NaCl (wt %) | 6.5 | 4-7 * |
| Physical form @ 25° C. | Clear, mobile liquid | Thick, firm paste |

* commercial product specification

These results demonstrated a surprising advantage for the inventive product. As it is was liquid at ambient temperature, storage and handling was greatly improved and no pre heating would be required for transfer operations.

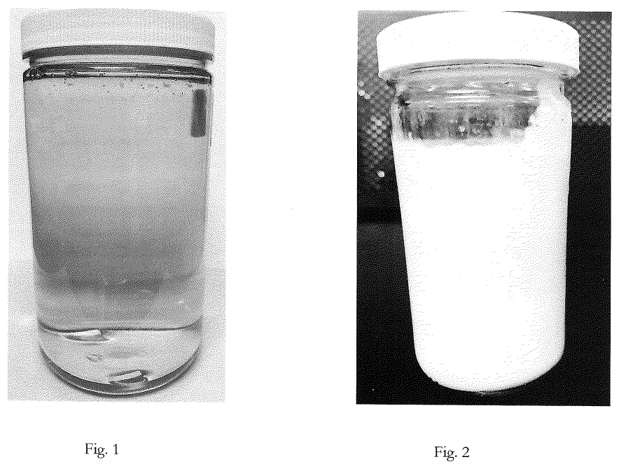

FIGS. 1 (Example 2, inventive) and 2 (Example 3, comparative) show the products at ambient temperature.

### Example 5—Physical Form of Isomeric Sodium Cocoyl N-methyl Methyl Taurate Mixture at Reduced Temperatures

This experiment was designed to simulate storage conditions in a cold warehouse.

A sample of the Example 2 product (100 g) was cooled in discrete steps. For each cooling step, the product was held for 30 minutes at that temperature, and the physical form of the product was visually inspected. The results are shown in Table 2.

TABLE 2
Physical form of Example 2 at reduced temperatures

| | |
|---|---|
| Temperature (° C.) | Physical form |

| | | |
|---|---|---|
| 5° | C. | Clear, thin liquid |
| 0° | C. | Clear, thin liquid |
| −5° | C. | Clear liquid, becoming more viscous |
| −10° | C. | Some solids apparent |
| −15° | C. | Sample solidified |

This experiment showed that inventive product of Example 2 remained fully liquid at temperatures as low as −5° C. This represents a significant advantage in commercial use as for most climates, the materials could be stored in non-temperature controlled warehousing.

The cooled Example 2 sample was then allowed to warm back to ambient temperature. The sample rapidly became fully liquid again, at a temperature of −8° C.

The present invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-11. (canceled)

12. A N-acylated taurine composition comprising:
(a) a first N-acylated taurine compound of the formula (IA) or a salt thereof: 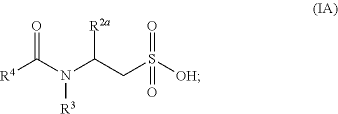
and (b) a second N-acylated taurine compound of the formula (IB) or a salt thereof: 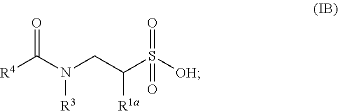
wherein
R^{1a} and R^{2a} are C₁₋₄ alkyl, wherein R^{1a} and R^{2a} are the same;
R³ is C₁₋₆ alkyl, C₂₋₆ alkenyl or C₁₋₆ alkyl substituted with an aryl group; and
R⁴ is C₄₋₂₅ alkyl or C₄₋₂₅ alkenyl, wherein the C₄₋₂₅ alkyl or C₄₋₂₅ alkenyl is optionally substituted by hydroxy.

13. The N-acylated taurine composition according to claim 1, wherein:
R^{1a} and R^{2a} are C₁₋₂ alkyl wherein R^{1a} and R^{2a} are the same;
R³ is C₁₋₆ alkyl; and
R⁴ is C₈₋₁₈ alkyl or C₈₋₁₈ alkenyl, wherein the C₈₋₁₈ alkyl or C₈₋₁₈ alkenyl is optionally substituted by hydroxy.

14. The N-acylated taurine composition according to claim 13, wherein R^{1a} and R^{2a} are methyl and/or R³ is C₁₋₂ alkyl.

15. The N-acylated taurine composition according to claim 1, comprising the first N-acylated taurine compound of the formula (IA) or a salt thereof and the second N-acylated taurine compound of the formula (IB) or a salt thereof in a molar ratio of 99:1 to 1:99, for example 99:1 to 1:1, for example 99:1 to 4:1.

16. The N-acylated taurine composition according to claim 15, comprising the first N-acylated taurine compound of the formula (IA) or a salt thereof and the second N-acylated taurine compound of the formula (IB) or a salt thereof in a molar ratio of 99:1 to 1:1 or a molar ratio of 99:1 to 4:1.

17. A method of preparing the N-acylated taurine composition according to claim 1, the method comprising reacting a taurine composition comprising a first taurine compound of the formula (IIA) or a salt thereof and a second taurine compound of the formula (IIB) or a salt thereof: 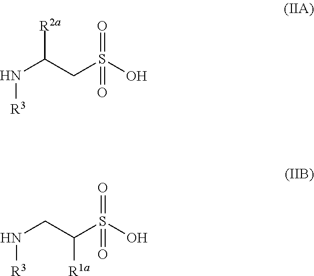
with a fatty acid of the formula R⁴—C(O)OH or a reactive derivative thereof, wherein R^{1a}, R^{2a}, R³ and R⁴ are each as defined in claim 12, to provide the N-acylated taurine composition.

18. The method according to claim 17, further comprising preparing the taurine composition by reacting an isethionic acid composition comprising a first isethionic acid compound of the formula (IIIA) or a salt thereof and a second isethionic acid compound of the formula (IIIb) or a salt thereof: 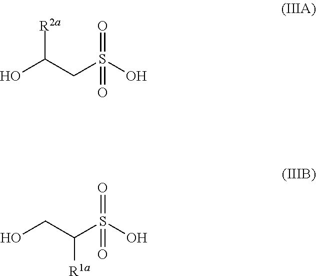
with a primary amine compound of the formula H₂NR³, wherein R^{1a}, R^{2a} and R³ are each as defined in claim 12, to provide the taurine composition.

19. The method according to claim 18, further comprising preparing the isethionic acid composition by reacting a source of bisulfite anions with an alkylene oxide of the formula (IV): 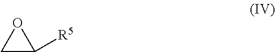
wherein R⁵ is the same as R^{1a} and R^{2a} as defined in claim 12.

20. A N-acylated taurine compound of the formula (IC) or a salt thereof: 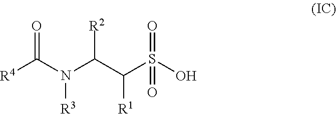
wherein:
R¹ and R² are each independently selected from H or C₁₋₄ alkyl, provided that one of R¹ and R² is H and the other of R¹ and R² is C₁₋₄ alkyl;
R³ is C₁₋₆ alkyl, C₂₋₆ alkenyl, or C₁₋₆ alkyl substituted with an aryl group; and
R⁴ is C₄₋₂₅ alkyl or C₄₋₂₅ alkenyl wherein the C₄₋₂₅ alkyl or C₄₋₂₅ alkenyl is optionally substituted by hydroxy.

21. The N-acylated taurine compound of the formula (IC) according to claim 20, wherein:
R¹ and R² are each independently selected from H or C₁₋₂ alkyl, provided that one of R¹ and R² is H and the other of R¹ and R² is C₁₋₂ alkyl;
R³ is C₁₋₆ alkyl; and
R⁴ is C₈₋₁₈ alkyl or C₈₋₁₈ alkenyl, wherein the C₈₋₁₈ alkyl or C₈₋₁₈ alkenyl is optionally substituted by hydroxy.

22. The N-acylated taurine compound of the formula (IC) according to claim 21 wherein R⁴ and R² are each independently selected from H or methyl, provided that one of R⁴ and R² is H and the other of R⁴ and R² is methyl, and/or R³ is C₁₋₂ alkyl.

23. A taurine composition comprising a first taurine compound of the formula (IIA) or a salt thereof and a second taurine compound of the formula (IIB) or a salt thereof: 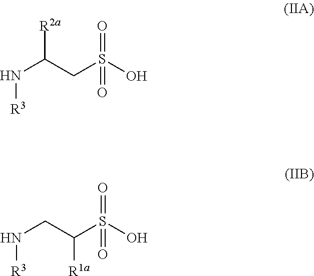
wherein
R^{1a} and R^{2a} are C₁₋₄ alkyl, wherein R^{1a} and R^{2a} are the same; and
R³ is C₁₋₆ alkyl, C₂₋₆ alkenyl or C₁₋₆ alkyl substituted with an aryl group.

24. A taurine composition according to claim 23, wherein:
R^{1a} and R^{2a} are C₁₋₂ alkyl, wherein R^{1a} and R^{2a} are the same; and
R³ is C₁₋₆ alkyl.

25. The taurine composition according to claim 24, wherein R^{1a} and R^{2a} are methyl and/or R³ is C₁₋₂ alkyl.

26. Use of a composition according to claim 12 in the formulation of a personal care, home care, industrial or agricultural product.

27. Use of a compound according to claim 20 in the formulation of a personal care, home care, industrial or agricultural product.

28. A method of making a product formulation selected from the group consisting of a personal care, home care, industrial or agricultural product comprising the step of utilizing the composition of claim 1 as a component of said product formulation.

29. A method of making a product formulation selected from the group consisting of a personal care, home care, industrial or agricultural product comprising the step of utilizing the compound of claim 9 as a component of said product formulation.
